(19) United States
(12) Patent Application Publication
(10) Pub. No.: US 2026/0283576 A1
(43) Pub. Date: Sep. 24, 2026

(54) Intra-Oral X-Ray Collimator Aiming System

(71) Applicant: Pac-Dent, Inc., Brea, CA (US)

(72) Inventors: Daniel Wang, Brea, CA (US); Kevin Wu, Brea, CA (US); Taosheng Hu, Suzhou (CN)

(73) Assignee: Pac-Dent, Inc., Brea, CA (US)

(21) Appl. No.: 19/573,336

(22) Filed: Mar. 20, 2026

Related U.S. Application Data
(60) Provisional application No. 63/775,110, filed on Mar. 20, 2025.

(51) Int. Cl. A61B 6/51

(52) U.S. Cl. CPC A61B 6/512

(57) ABSTRACT

A dental X-ray aiming system. The system includes an aiming ring, a sensor support arm and a sensor holder configured to hold an image sensor in the X-ray field of a dental X-ray system, and a radiopaque shield disposed with the aiming ring. The shield includes an oblong aperture which allows passage of X-rays through the ring. The shield is rotatable relative to the sensor, in order to align a long axis of the aperture with the long axis of the sensor, thus blocking stray X-rays which represent unneeded exposure to the patient and which may degrade the quality of the image with stray X-rays.

### CROSS-REFERENCE TO RELATED APPLICATIONS

This application claims priority to U.S. Provisional Application 63/775,110, filed Mar. 20, 2025, pending.

### FIELD OF THE INVENTIONS

The inventions described below relate to the field of dental X-ray equipment.

### BACKGROUND OF THE INVENTIONS

X-ray aiming systems for dental X-rays are used to properly locate X-ray sensors within the mouth of a patient and in line with an X-ray collimator tube. These systems generally include an aiming ring matched to the collimator tube, a support arm configured to hold a sensor in the mouth of a patient, and a sensor holder which can support the sensor on the end of the support arm. The use of the collimator tube limits exposure of the patient to X-rays, eliminating, focusing or blocking stray X-rays not aimed at the patient’s mouth. Abromovich, et al., Dental Positioning System, U.S. Patent 9,314,215 (Apr. 19, 2016), for example, discloses a dental x-ray collimator aiming system which includes an aiming ring, a support arm tab attached to the aiming ring, with several apertures keyed to the cross-sections of a variety of support arms. This system uses a shield within the aiming ring, with a symmetrical aperture in the shield.

### SUMMARY

The devices and methods described below provide for obtaining dental X-rays while minimizing exposure of the patient. The aiming system includes an aiming ring, a sensor support arm and a sensor holder (at least one of each) configured to hold an image sensor in the X-ray field of a dental X-ray system. The system includes a radiopaque shield disposed within the aiming ring, and this shield includes an oblong aperture which allows passage of X-rays through the ring. The shield is rotatable relative to the sensor, in order to align a long axis of the aperture with the long axis of the sensor, thus blocking stray X-rays which represent unneeded exposure to the patient and which may degrade the quality of the image with stray X-rays.

### BRIEF DESCRIPTION OF THE DRAWINGS

FIGS. 1, 2 and 3 illustrate components of the Intra-Oral X-Ray Collimator Aiming System.

FIGS. 4, 5 and 6 illustrate sensor holders configured to hold a sensor inside a patient’s mouth.

FIGS. 7, 7A, 8 and 9 illustrate the Intra-Oral X-Ray Collimator Aiming System with several support arms and corresponding sensor holders assembled with the aiming ring.

FIGS. 10, 10A, 11 and 12 show the system installed in a typodont, to illustrate placement and use of the aiming ring and sensor holder in the various configurations illustrated in FIGS. 7, 7A, 8 and 9.

### DETAILED DESCRIPTION OF THE INVENTIONS

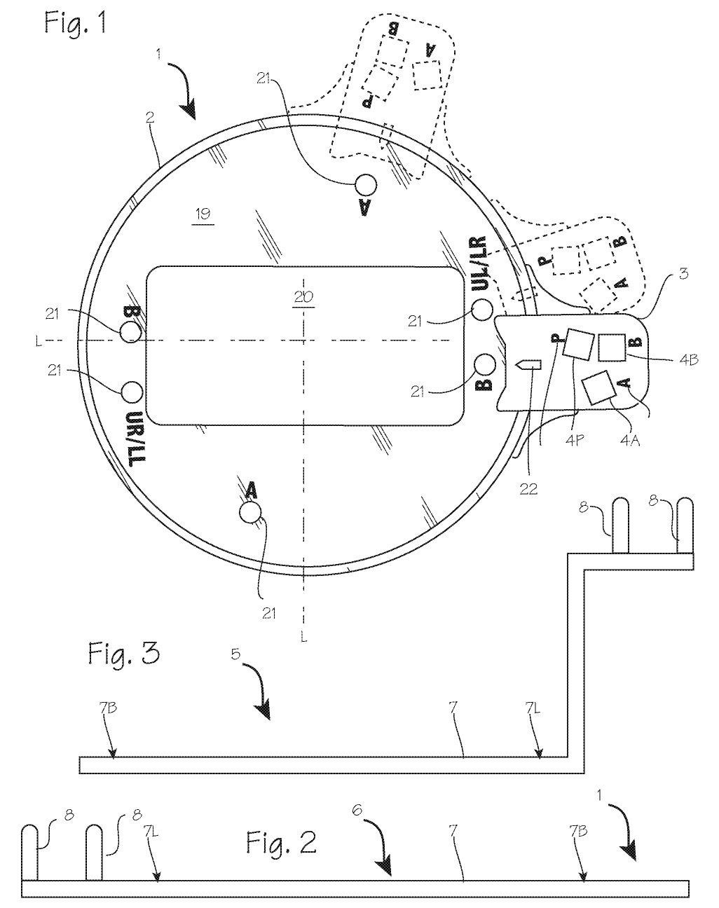

FIGS. 1, 2 and 3 illustrate the Intra-Oral X-Ray Collimator Aiming System. The aiming system includes the aiming ring assembly 1 shown in FIG. 1, which includes the aiming ring 2 and support arm tab 3 with one or more support arm receiving apertures 4A for receiving a support arm for an anterior image, 4B for receiving a support arm for a bitewing image, and 4P for receiving a support arm for a posterior image (which might be upper right (UR), lower left (LL), upper left (UL) or lower right (LR) posterior images).

The aiming ring 2 is sized to accommodate the end of an X-ray collimator tube. The collimator tube serves to narrow X-rays to cause the X-rays to travel along, parallel or almost parallel to the axis of the tube (rather than broadcast in all directions) and also to limit the width of the beam passing into a patient. Thus, the diameter of the ring closely matches the diameter of a collimator tube of an X-ray imaging system with which it is to be used, so that a technician can place the emitting end of the collimator tube close to the aiming ring and with the long axis of the tube aligned with the axis of the ring passing through the ring and a sensor held by the aiming assembly such that X-rays passing through the ring and aperture pass through the sensor.

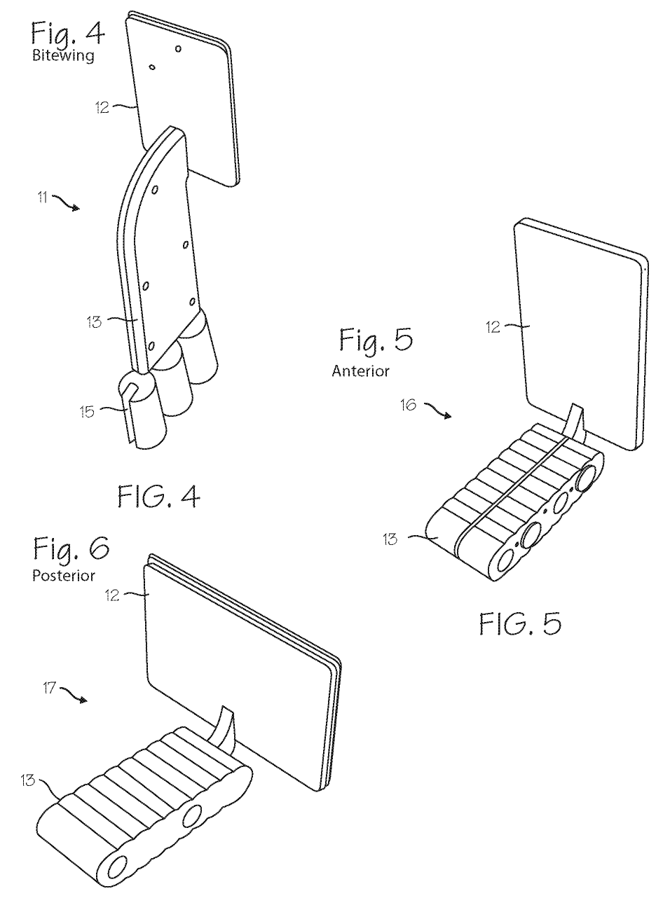

A support arm is a bar configured to hold a sensor holder in a field of view established by the aiming ring assembly. In the illustrated system, two support arms are used. The support arms are configured to hold sensor holders in a field of view established by the aiming ring assembly and are shown in FIGS. 2 and 3. The support arm 5 of FIG. 2 is configured to hold a sensor holder in position relative to the aiming ring to capture a bitewing image. The support arm 6 of FIG. 3 is configured to hold a sensor holder in position relative to the aiming ring to capture an anterior image or a posterior image. Each support arm comprises a distancing rod 7 with a first end 7B (a proximal end that remains outside the mouth, on the buccal or facial side of the teeth) configured to fit within an aperture of the support arm tab 3 and a second end 7L (a distal end that is positioned in the mouth on the lingual side of the teeth) with sensor holder support pins 8 disposed on the second end, with the pins configured to hold a sensor in an orientation, relative to the aiming ring, in the field of exposure of an X-ray emitter and X-rays passing through the aiming ring. The support arm 5 of FIG. 2 is a bitewing support arm configured to support a bitewing sensor holder shown below such that a sensor attached to the holder may be disposed interior to a patient’s bitewing while the X-ray emitter is aimed at the bitewing. The support arm 6 of FIG. 3 is an anterior/posterior support arm configured to hold an anterior and/or a posterior sensor holder shown below such that a sensor 9 attached to the holder may be disposed interior to a patient’s teeth, upper or lower. The anterior/posterior support arm can be used with a sensor holder configured for placement behind the front teeth (anterior) or a sensor holder configured for placement behind the molars/back teeth (posterior). More varied support arms may be used, configured for use of complementary sensor holders, or a single support arm may be used in conjunction with sensor holders configured to support an imaging plate in the field of exposure. Each sensor holder, illustrated in FIGS. 4, 5 and 6, is configured to hold an image sensor within the mouth of a patient opposite a collimator aligned with an aiming ring. 10

Bitewing: FIG. 4 shows a bitewing sensor holder 11 with a sensor plate 12 configured to hold an X-ray sensor on sensor plate, a bite block 13 configured to be trapped between a patient’s front teeth, and a connecting stem 14 holding the sensor plate on the bite block, and a base 15 with apertures for fixing the bitewing sensor holder to the support arm such that the long axis of the sensor plate, and thus a rectangular sensor attached to the plate, is oriented along an anterior-posterior line (front to back) relative to the patient, and the sensor plate is inside the patient’s mouth between the tongue and the lingual surface of both top and bottom back teeth, to obtain a bitewing image of the patient’s back teeth.

Anterior: FIG. 5 shows an anterior sensor holder 16 configured to hold an X-ray sensor on sensor plate 12 when the bite block 13 is trapped between a patient’s front teeth, such that the long axis of the sensor plate, and thus a rectangular sensor, is oriented along a superior-to-inferior line (top to bottom) relative to the patient, and the sensor plate is inside the patient’s mouth proximate the lingual surface of a front tooth, to obtain an anterior image of a top or bottom front tooth.

Posterior: FIG. 6 shows a posterior sensor holder 17 configured to hold an X-ray sensor on sensor plate 12 when the bite block 13 is trapped between a patient’s back teeth, such that the long axis of the sensor plate, and thus a rectangular sensor, is oriented along an anterior-to-posterior line (front to back) relative to the patient, and the sensor plate is inside the patient’s mouth proximate the lingual surface of a back tooth to obtain a posterior image of a top or bottom back tooth.

Each of the sensor plates 12 is preferably rectangular (to aid in proper placement of typically rectangular X-ray holders) and characterized by a long axis and a short axis. Each of the sensor holders and support arm second ends includes cooperative means for releasable attachment of the holder to the second end of the support arm, which, as illustrated, comprise pins 8 on the second end and pin receiving channels 18 on a first end of the sensor holder (the first end is opposite the second end which includes the sensor plate 12). Other suitable releasable attachment means may be used. For example, the pins may be disposed on the sensor holder and the apertures on the support arms, or a single keyed pin (or a non-circular cross-section of the support arm) may be keyed to a non-circular aperture in the holder (or vice-versa), or a cooperating mortise and tenon arrangement may be used.

Each of the support arm first ends 7B together with the apertures 4A, 4B, and 4UR includes cooperative means for releasable attachment of the first ends 7B to the support arm tab 3, which, as illustrated, comprise the aperture with a non-circular cross-section keyed to a non-circular cross-section of the first ends 7B, such that the support arms cannot rotate relative to the aperture or support arm tab. Other releasable non-rotatable cooperative means for releasable attachment may be used to secure the support arms to the support arm tab. For example, pins may be disposed on the support arm first end to mate with apertures on the support tab, or a single keyed pin (or a non-circular cross-section of the support arm) may be keyed to a non-circular aperture in the support tab (or vice-versa), or a cooperating mortise and tenon arrangement may be used, or clips may be used.

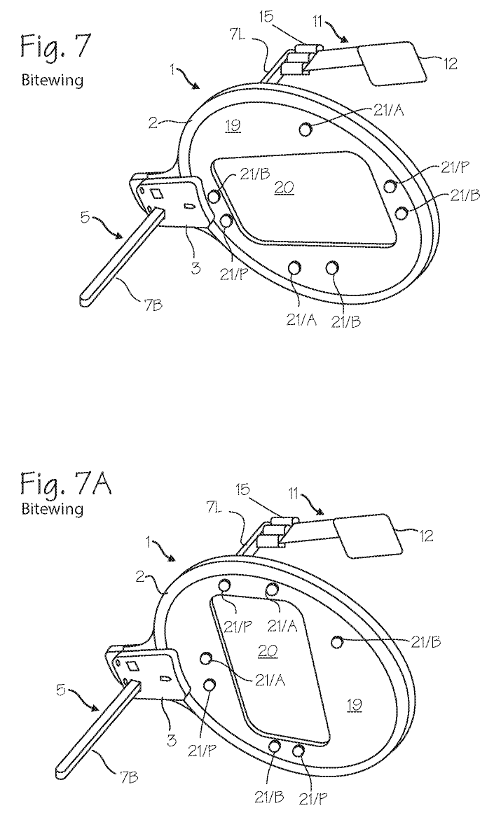

FIG. 7, 7A, 8 and 9 illustrates different configurations of the Intra-Oral X-Ray Collimator Aiming System with the support arm and sensor holders secured to the aiming ring.

As shown in FIG. 7, the distancing rod 7 first end 7B is secured to the support arm tab 3 (and thus secured to the ring 2), and is inserted into the support arm receiving aperture 4B (or securing the bitewing support arm to the support arm tab 3 through other cooperative means for releasable attachment). The bitewing sensor holder 11 is fitted onto the bitewing support arm 5 at the second end 7L of the distancing rod 7 by slipping the pins 8 into the holes in the base 15 of the bite block 13 of the bitewing sensor holder 11 (or securing the holder to the support arm second end through other cooperative means for releasable attachment). This configures the assembly for placement on the patient, with the sensor plate and a sensor on the plate in the mouth of the patient for a bitewing image and the aiming ring outside the mouth with the ring aligned with the sensor plate such that X-rays exiting a collimator tube (aligned with the aiming ring 2 such that the long axis of the collimator tube is aligned with an axis of the ring (passing through the ring) and the sensor plate (typically, an axis perpendicular to the plane established by the ring) are, in the area occupied by the sensor plate, not blocked and thus pass through the aperture, while X-rays directed to areas peripheral to the sensor plate are blocked by the shield (and thus do not reach the patient, thereby reducing X-ray exposure for the patient). In FIG. 7, the long axis of the shield aperture 20 is aligned with the long axis of the sensor plate 12. As shown in FIG. 7A, vertical bitewing images may be obtained with the support arm and sensor holder in the same positions, but with the ring and shield 19 and aperture shield aperture 20 rotated 90˚ (relative to FIG. 7) so that the long axis of the shield aperture 20 is aligned with the short axis of sensor plate 12.

FIG. 8 illustrates the Intra-Oral X-Ray Collimator Aiming System with the anterior/posterior support arm 6 and posterior sensor holder secured to the aiming ring. As shown in this figure, the anterior/posterior support arm 6 distancing rod 7 first end 7B is secured to the support arm tab 3 (and thus secured to the ring 2), and is inserted into the support arm receiving aperture 4A (or securing the anterior/posterior support arm 6 to the support arm tab 3 through other cooperative means for releasable attachment) and the anterior sensor holder 16 is fitted onto the anterior/posterior support arm 6 distancing rod second end 7L by slipping the pins 8 into the holes in the bite block 13 of the anterior sensor holder 16 (or securing the holder to the support arm second end through other cooperative means for releasable attachment). This configures the assembly for placement on the patient, with the sensor plate and a sensor on the plate in the mouth of the patient for an anterior image and the aiming ring outside the mouth with the ring aligned with the sensor plate such that X-rays exiting a collimator tube (aligned with the aiming ring 2 such that the long axis of the collimator tube is aligned with an axis of the ring (passing through the ring) and the sensor plate (typically, an axis perpendicular to the plane established by the ring) are, in the area occupied by the sensor plate, not blocked and thus pass through the aperture, while X-rays directed to areas peripheral to the sensor plate are blocked by the shield (and thus do not reach the patient, thereby reducing X-ray exposure for the patient.

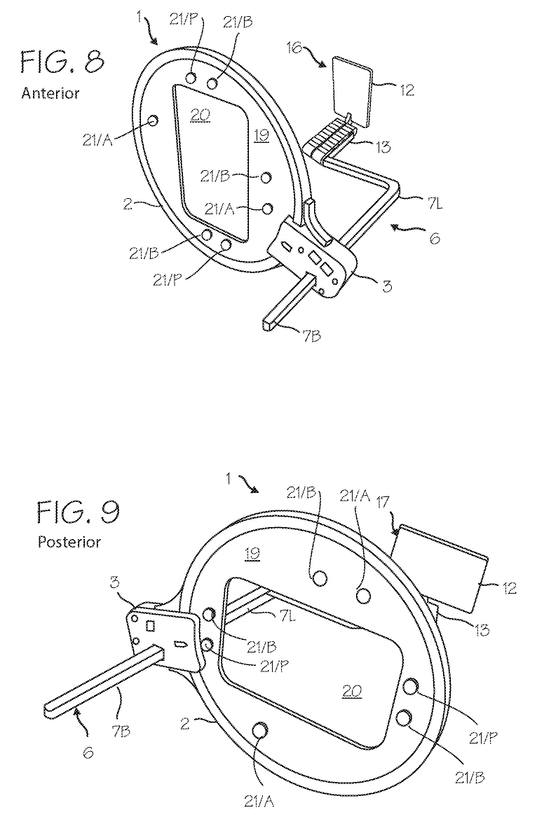

FIG. 9 illustrates the Intra-Oral X-Ray Collimator Aiming System with the posterior support arm and posterior sensor holder secured to the aiming ring. As shown in this figure, the anterior/posterior support arm 6 distancing rod 7 first end 7B is secured to the support arm tab 3 (and thus secured to the ring 2), and is inserted into the support arm receiving aperture 4UR (or securing the posterior support arm to the support arm tab 3 through other cooperative means for releasable attachment) and the posterior sensor holder 17 is fitted onto the anterior/posterior support arm 6 distancing rod 7 second end 7L by slipping the pins 8 into the holes in the bite block of the posterior sensor holder 17 (or securing the holder to the support arm second end through other cooperative means for releasable attachment). This configures the assembly for placement on the patient, with the sensor plate and a sensor on the plate in the mouth of the patient for a posterior image and the aiming ring outside the mouth with the ring aligned with the sensor plate such that X-rays exiting a collimator tube aligned with the aiming ring 2 such that the long axis of the collimator tube is aligned with an axis of the ring passing through the ring and the sensor plate (typically, an axis perpendicular to the plane established by the ring).

To limit the amount of X-rays passing through the aiming ring assembly 1 to the patient, a shield 19 is disposed with the aiming ring 2. The shield comprises a radiopaque material (typically, metal). An aperture 20 allows X-rays to pass through the shield only through the aperture. The shield is planar and lies in a plane established by the aiming ring. The aperture is oblong (rectangular or oval, for example, and may be symmetrical or asymmetrical) with a long axis L and a short axis S. The shield is rotatable, about the axis passing through the aiming ring, to block X-rays from exposing areas of the patient spaced from the sensor and thus serving no imaging purpose yet increases X-ray exposure for the patient. The support arm tab 3 may be revolved about the central axis of the aiming rim (that is, translated along the rim of the aiming ring) or, conversely, the aiming ring and/or shield may be rotated about a central axis while the support arm tab 3 remains fixed relative to the central axis, or the shield may be rotated within the aiming ring, to align the long axis of the aperture with the long axis of a sensor fixed to the sensor plate held by the support arm and sensor holder. To allow for certain alignment of the long axis of the aperture with the long axis of the sensor and/or sensor plate, various indicia may be included on the aiming ring assembly such as the depicted indicia including letters A, B, UR/LL, UL/LR, and color-coded symbols (in this case, dots) 21. Preferably, the dots corresponding the desired position for anterior images are blue and labeled with the letter A, the dots corresponding the desired position for posterior images are yellow and labeled with the letters UR/LL, UL/LR, and the dots corresponding the desired position for bitewing images are red and labeled with the letter B.

A pointer 22 on the support arm tab 3 (any suitable indicia (letters, symbols, other markings such as circles, dots, etc.)), and indicia A (for anterior) UR/LL (for posterior) and B (for bitewing) are marked on the rim or shield (preferably the shield, as shown). Corresponding indicia can be used to mark the support arm receiving apertures 4, to indicate which aperture will accommodate the bitewing support arm in proper position, the anterior support arm, or the posterior support arm. A technician placing the aiming ring on a patient can install the bitewing support arm into the aperture B, or install the anterior support arm into the aperture A, or the posterior support arm into aperture B, and revolve/translate the support arm tab (or rotate the shield within the ring) to locate the indicia 22 on the support arm tab 3 corresponding to the selected support arm in opposition to the indicia on the shield or ring corresponding to the selected support arm. The support arm tab 3 may be slid about the rim (with the shield rotationally fixed to the rim), or the shield may be rotated within the rim (with the rim fixed to the support tab so that it cannot slide along the rim), to accomplish the step of rotating the shield relative to the support tab (or, conversely, translate the support tab circumferentially about the shield). Indicia may also be color-coded, with red indicia used to signify components, sensor holders and apertures for bitewing imaging, blue indicia used to signify components, sensor holders and apertures for vertical anterior imaging, and yellow indicia used to signify components, sensor holders and apertures for horizontal posterior imaging.

In use, a technician will assemble the aiming ring assembly 1 by inserting a first end (7B) of a sensor support arm (bitewing support arm 5 or anterior/posterior support arm 6) into one of the apertures in the support arm tab 3, install a sensor holder (11, 16 or 17) on the second end (7L) of the chosen sensor support arm, and translate the support tab around the ring (or rotate the shield relative to the support tab) to align the long axis of the aperture with the long axis of the support plate or sensor. The technician will then place the assembled aiming ring assembly, with the sensor secured to the sensor plate, on the patient, with the sensor plate and sensor in the mouth of the patient, the bite block between the patient’s upper and lower teeth, and the aiming ring outside the patient’s mouth. Once the sensor plate and sensor are secured in place, with the shield aperture aligned, the technician may expose the patient and sensor to X-rays passed through the aperture of the aiming ring to obtain images.

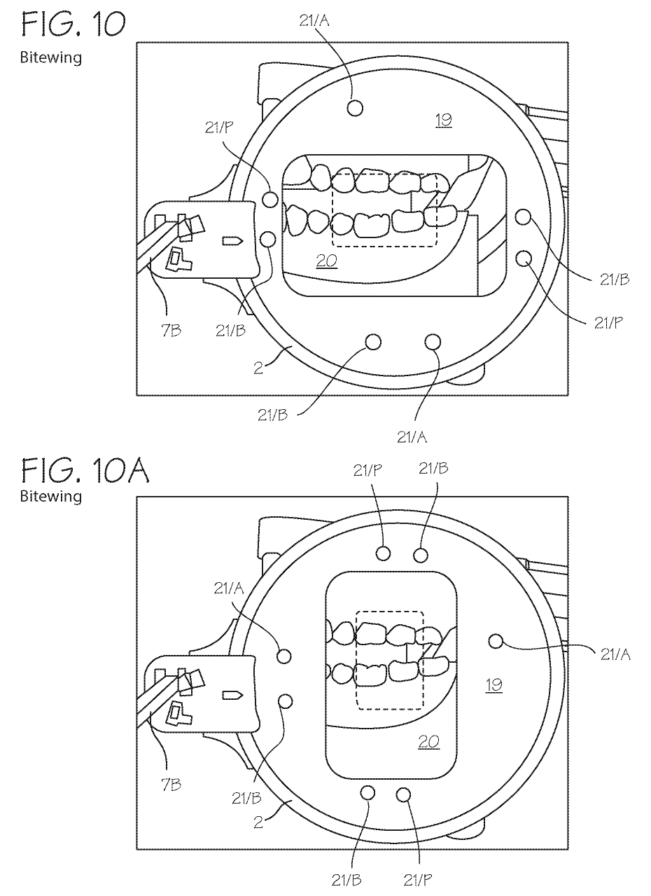

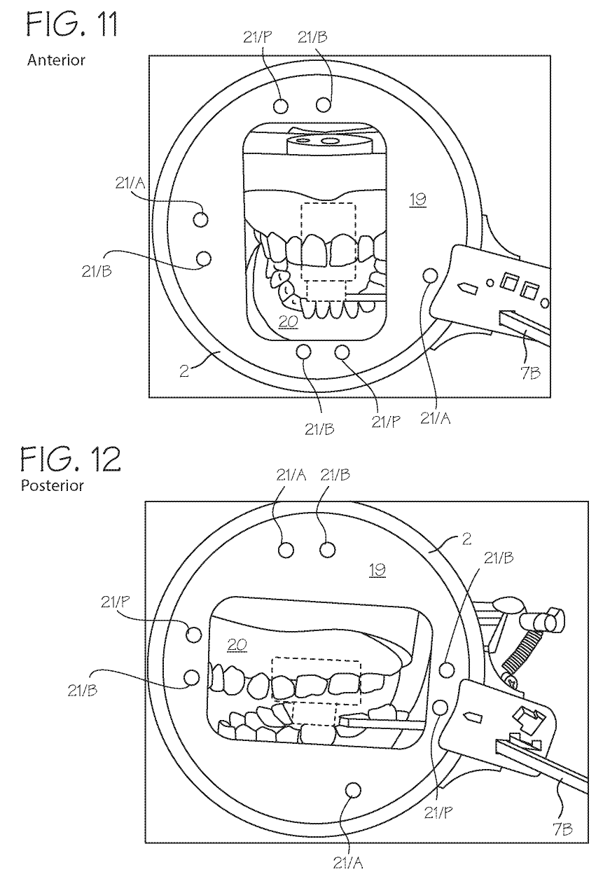

FIGS. 10, 10A, 11 and 12 show the system installed in a typodont, to illustrate placement and use of the aiming ring and sensor holder in the various configurations illustrated in FIGS. 7, 8 and 9. FIG. 10 illustrates the placement of the system as configured in FIG. 7, with the image sensor 9 (phantom line) inside the back teeth, behind the sensor plate, and the mounting tab translated about the ring to locate the window of the shield such that the long axes of both are aligned (parallel, or substantially parallel). This configuration is useful to obtain a horizontal bitewing image. FIG. 10A illustrates the placement of the system as configured in FIG. 7A, with the image sensor 9 (phantom line) inside the back teeth (in a vertical orientation) and the mounting tab translated about the ring to locate the window of the shield such that the long axes of both are aligned with each other (parallel, or substantially parallel). This configuration is useful to obtain a vertical bitewing image. FIG. 11 illustrates the placement of the system as configured in FIG. 8, with the image sensor 9 (phantom line) inside the front teeth and the mounting tab translated about the ring to locate the window of the shield such that the long axes of both are aligned (parallel, or substantially parallel). This configuration is useful to obtain an anterior image. FIG. 12 illustrates the placement of the system as configured in FIG. 9, with the image sensor 9 (phantom line) inside the back upper teeth and the mounting tab translated about the ring to locate the window of the shield such that the long axes of both are aligned (parallel, or substantially parallel). This configuration is useful to obtain a posterior image. Note that each of these configurations may be achieved, if the mounting tab is fixed to the ring (not translatable about the ring) and the shield is rotatable within the ring, by rotating the shield to place the pointer such that it is aligned (pointing to) indicia corresponding to the sensor holder and the desired image.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A dental aiming ring assembly for use with a dental X-ray image sensor and a collimator of an X-ray imaging system, said system comprising:
an aiming ring (2);
a radiopaque shield (19) disposed within the aiming ring (2);
a sensor holder (11, 16, 17) configured to hold an image sensor (9) within the mouth of a patient;
a first support arm (5 or 6) configured to hold the sensor holder and image sensor in a first position spaced from the aiming ring (2); wherein
the radiopaque shield (19) has an oblong aperture (20), and said radiopaque shield is rotatable relative to the sensor holder (11, 16, 17).

2. The dental aiming ring assembly of claim 1, further comprising:
a support arm tab (3) secured to the aiming ring (2);
cooperative means for releasable attachment of a first end (7B) of the first support arm (5 or 6) to the support arm tab (3); wherein
the support arm tab (3) is slidable about the aiming ring (2), whereby the radiopaque shield (19) within the aiming ring (2) is rotatable relative to an image sensor (9) fixed to the sensor holder (11, 16, 17) when a first end (7B) of the first support arm (5 or 6) is attached to the support arm tab (3) and the first end (7B) of the first support arm (5 or 6), with the sensor holder (11, 16, 17) attached to the second end (7L) of the first support arm (5 or 6) and the image sensor (9) is attached to the sensor holder (11, 16, 17).

3. A dental aiming ring assembly of claim 1, further comprising:
a support arm tab (3) secured to the aiming ring (2); and
cooperative means for releasable attachment of a first end (7B) of the first support arm (5 or 6) to the support arm tab (3); wherein
the radiopaque shield (19) is rotatable within the aiming ring (2).

4. A dental aiming ring assembly of claim 1, wherein:
the aiming ring (2) is characterized by a plane in which the aiming ring lies; and
the oblong aperture (20) is characterized by a long axis and a short axis, and
the radiopaque shield (19) is rotatable in a plane parallel to or coincident with the plane of the aiming ring (2) to align the long axis of the aperture with a long axis of an image sensor secured to the sensor support.

5. A dental aiming ring assembly of claim 2, further comprising:
a second support arm (5 or 6) configured to hold a second sensor holder and an image sensor (9) in a second position spaced from the aiming ring (2); and
cooperative means for releasable attachment of a first end (7B) of the second support arm (5 or 6) to the support arm tab (3);
the cooperative means for releasable attachment of a first end (7B) of the first support arm (5 or 6) to the support arm tab (3) comprises an aperture in the support arm tab having a non-circular cross-section and a portion of the first end (7B) of the first support arm (5 or 6) with a non-circular cross-section keyed to the non-circular cross-section of the aperture such that the first support arm is rotationally fixed to the support arm tab;
the cooperative means for releasable attachment of a first end (7B) of the second support arm to the support arm tab (3) comprises a second aperture in the support arm tab having a non-circular cross-section and a portion of the first end (7B) of the second support arm with a non-circular cross-section keyed to the non-circular cross-section of the aperture such that the second support arm is rotationally fixed to the support arm tab.

6. A dental aiming ring assembly of claim 5, further comprising:
first indicia disposed on the support arm tab associated with the first aperture, said first indicia indicative of a first standard X-ray image to be obtained with the support arm attached to the support arm tab through the first aperture;
second indicia disposed on the support arm tab associated with the second aperture, said second indicia indicative of a second standard X-ray image to be obtained with the support arm attached to the support arm tab through the second aperture.

7. A dental aiming ring assembly for use with a dental X-ray image sensor and a collimator of an X-ray imaging system, said system comprising:
an aiming ring (2);
a radiopaque shield (19) disposed within the aiming ring (2);
a sensor holder (11, 16, 17) configured to hold an image sensor (9) within the mouth of a patient;
an image sensor (9) fixed to the sensor holder;
a first support arm (5 or 6) coupled to the sensor holder and configured to hold the image sensor in a first position spaced from the aiming ring (2); wherein
the radiopaque shield (19) has an oblong aperture (20), and said radiopaque shield is rotatable relative to the sensor holder (11, 16, 17) and the image sensor (9) fixed to the sensor holder.

8. A method of obtaining dental X-ray images, said method comprising the steps of:
providing a dental aiming ring system for use with a dental X-ray image sensor and a collimator of an X-ray imaging system, said system comprising:
an aiming ring (2);
a radiopaque shield (19) disposed within the aiming ring (2);
a sensor holder (11, 16, 17) configured to hold the sensor holder (11, 16, 17) within the mouth of a patient;
an oblong image sensor (9) secured to the sensor holder (11, 16, 17);
a first support arm (5 or 6) configured to hold the sensor holder (11, 16, 17) and image sensor (9) in a first position spaced from the aiming ring (2); wherein
the radiopaque shield (19) has an oblong aperture (20), and said radiopaque shield is rotatable relative to the image sensor (9) fixed to the sensor holder (11, 16, 17);
rotating the radiopaque shield (19) to align the oblong aperture (20) with the oblong image sensor (9) secured to the sensor holder (11, 16, 17); and
obtaining X-ray images on the image sensor (9) by directing X-rays through the aiming ring (2) and aperture (20) and toward the image sensor (9).
